(19) United States
(12) Patent Application Publication
(10) Pub. No.: US 2024/0390058 A1
(43) Pub. Date: Nov. 28, 2024

(54) RECONFIGURABLE MULTI-PURPOSE SURGICAL TOOL SYSTEMS, METHODS, AND DEVICES

(71) Applicant: THE BOARD OF REGENTS OF THE UNIVERSITY OF TEXAS SYSTEM, Austin, TX (US)

(72) Inventors: Shelby Richard Lies, Dallas, TX (US); Robert Reinhardt, Dallas, TX (US); Rohan Selvaraj, Dallas, TX (US); Joel Johnson, Dallas, TX (US); Antonio Cadena, Dallas, TX (US); Jonathan Mondragon, Dallas, TX (US); Hirak Patel, Dallas, TX (US)

(21) Appl. No.: 18/674,529

(22) Filed: May 24, 2024

Related U.S. Application Data
(60) Provisional application No. 63/504,035, filed on May 24, 2023.

(51) Int. Cl. A61B 18/14; A61B 18/00

(52) U.S. Cl. CPC A61B 18/1445; A61B 2018/00178; A61B 2018/00595; A61B 2018/00607; A61B 2018/1455

(57) ABSTRACT

Systems, methods, and devices include a reconfigurable multi-purpose surgical tool with a housing having a plurality of portions for a plurality of surgical tools, such as a cautery tool, a scalpel tool, and/or a forceps tool. A first portion of the housing can house a first surgical tool. The first surgical tool is converted between an in-use configuration and a stored configuration. The stored configuration includes a first surgical tool portion of the first surgical tool being at least partially contained by the housing. The in-use configuration being when the first surgical tool portion is at least partially exposed from the housing. A second portion of the housing partially houses a second surgical tool. The second surgical tool includes another in-use configuration with the second surgical tool at least partially exposed from the housing. A third portion of the housing at least partially houses a third surgical tool.

### CROSS REFERENCE TO RELATED APPLICATION

This application claims priority to U.S. Provisional Patent Application Ser. No. 63/504,035, filed May 24, 2023, and titled “RECONFIGURABLE MULTI-PURPOSE SURGICAL TOOL SYSTEMS, METHODS, AND DEVICES,” which is incorporated by reference herein in its entirety.

### FIELD

The present disclosure relates to surgical tools and more particularly to housings for surgical tools.

### BACKGROUND

Surgical procedures often involve many tools at different stages of the procedure. Managing the usage and distribution of the surgical tools between the surgeons and other medical staff while maintaining a safe sterile environment can be challenging. It is with these observations in mind, among others, that various aspects of the present disclosure were conceived and developed.

### BRIEF SUMMARY

The presently disclosed technology addresses the foregoing problems by providing systems, methods, and devices including a reconfigurable multi-purpose surgical tool. The reconfigurable multi-purpose surgical tool can include a housing and a first portion of the housing operable to at least partially house a first surgical tool. The first surgical tool can be operable to be selectively converted between a first plurality of configurations, the first plurality of configurations including an in-use configuration and a stored configuration. The stored configuration can include a first surgical tool portion of the first surgical tool being at least partially contained by the housing, and the in-use configuration can be when the first surgical tool is at least partially exposed from the housing. Also, a second portion of the housing can be operable to at least partially house a second surgical tool, the second surgical tool being operable to include at least another in-use configuration. The another in-use configuration can be when the second surgical tool is at least partially exposed from the housing.

In some examples, the first portion of the housing can be a cautery housing section, and the first surgical tool can be a cautery tool. The second portion of the housing can be a forceps housing section, and the second surgical tool can be a forceps tool. Furthermore, the reconfigurable multi-purpose surgical tool can include a third portion of the housing operable to at least partially house a scalpel tool. The first surgical tool can extend from an opposite end of the housing relative to the second surgical tool. Moreover, the first surgical tool can be operable to be in the in-use configuration when the second surgical tool is in the another in-use configuration. Additionally, the second portion of the housing can include an internal mounting surface for positioning the second surgical tool in the second portion of the housing. The reconfigurable multi-purpose surgical tool can further include a tool slider, with a locking mechanism, for converting the first surgical tool between the first plurality of configurations.

In some instances, the reconfigurable multi-purpose surgical tool includes an electrical connector configured to connect at least one of the first surgical tool or the second surgical tool to an electrosurgical generator. Also, the first portion of the housing can be a cautery housing section, the first surgical tool can be a cautery tool, the second portion of the housing can be a scalpel housing section, and/or the first surgical tool can be a scalpel tool. Furthermore, the second surgical tool can be operable to be selectively converted between a second plurality of configurations. The second plurality of configurations can include the another in-use configuration and another stored configuration, the another stored configuration including a second surgical tool portion of the second surgical tool being at least partially contained by the housing.

In some examples, a reconfigurable multi-purpose surgical tool includes a housing and a first portion of the housing operable to at least partially house a first surgical tool. The first surgical tool can be operable to be selectively converted between a first plurality of configurations, and the first plurality of configurations can include an in-use configuration and a stored configuration. The stored configuration can include a first surgical tool portion of the first surgical tool being at least partially contained by the housing. The in-use configuration can be when the first surgical tool portion is extended at least partially from a first end of the housing. Furthermore, the reconfigurable multi-purpose surgical tool can include a second portion of the housing operable to at least partially house a second surgical tool, the second surgical tool, having a configuration extending from a second end of the housing opposite the first end.

In some instances, the first portion of the housing can be a cautery housing or a scalpel housing, the first surgical tool can be a cautery tool or a scalpel tool, the second portion of the housing can be a forceps housing, and/or the first surgical tool can be a forceps tool. Additionally or alternatively, the first surgical tool is a cautery tool, and the reconfigurable multi-purpose surgical tool also includes a slider assembly with a cautery tool locking member for engaging with a slide channel along the housing. Moreover, the reconfigurable multi-purpose surgical tool can include a third surgical tool being a scalpel tool, and/or a second slider assembly coupled to the scalpel tool for moving the scalpel tool between an extended position and a retracted position, the second slider assembly including a second tool locking member for engaging with a second slide channel along the housing. The third surgical tool can also be extendible, from the first end of the housing, in a direction parallel to the first surgical tool.

In some examples, a reconfigurable multi-purpose surgical tool includes a housing. A first portion of the housing can be operable to at least partially house a first surgical tool, and/or the first surgical tool operable to be selectively converted between a first plurality of configurations, the first plurality of configurations including an in-use configuration and a stored configuration. The stored configuration can include a first surgical tool portion of the first surgical tool being at least partially contained by the housing. Moreover, the in-use configuration can be when the first surgical tool is at least partially exposed. The reconfigurable multi-purpose surgical tool can also include a second portion of the housing operable to at least partially house a second surgical tool. The second surgical tool can be operable to be selectively converted between a second plurality of configurations, the second plurality of configurations including another in-use configuration and another stored configuration. The another stored configuration can include a second surgical tool portion of the second surgical tool being at least partially contained by the housing. Additionally, the another in-use configuration can be when the second surgical tool portion is at least partially exposed at a same end of the housing as the first surgical tool.

In some instances, the first portion of the housing can be a cautery housing section, the first surgical tool can be a cautery tool, the second portion of the housing can be a scalpel housing section, and/or the first surgical tool can be a scalpel tool. The reconfigurable multi-purpose surgical tool can also include one or more power buttons disposed at a sidewall of the cautery housing section of the housing operable to control a power supply to a control circuit of the cautery tool. Additionally, the reconfigurable multi-purpose surgical tool can include a third portion of the housing operable to at least partially house a third surgical tool being a forceps tool. Furthermore, the reconfigurable multi-purpose surgical tool can include an actuator button disposed at a side wall of the third portion of the housing operable to apply a compressive force to the forceps tool.

The foregoing is intended to be illustrative and is not meant in a limiting sense. Many features of the examples may be employed with or without reference to other features of any of the examples. Additional aspects, advantages, and/or utilities of the presently disclosed technology will be set forth in part in the description that follows and, in part, will be apparent from the description, or may be learned by practice of the presently disclosed technology.

### BRIEF DESCRIPTION OF THE DRAWINGS

The foregoing summary, as well as the following detailed description, will be better understood when read in conjunction with the appended drawings. For the purpose of illustration, there is shown in the drawings certain examples of the disclosed subject matter. It should be understood, however, that the disclosed subject matter is not limited to the precise examples and features shown. The accompanying drawings, which are incorporated in and constitute a part of this specification, illustrate an implementation of systems and methods consistent with the disclosed subject matter and, together with the description, serves to explain advantages and principles consistent with the disclosed subject matter, in which:

FIG. 1 illustrates an example system including a reconfigurable multi-purpose surgical tool with a cautery housing section, a scalpel housing section, and/or a forceps housing section;

FIG. 2 illustrates an example system including a cautery housing section of a reconfigurable multi-purpose surgical tool, which can form at least a portion of the system depicted in FIG. 1;

FIG. 3 illustrates an example system including a scalpel housing section of a reconfigurable multi-purpose surgical tool, which can form at least a portion of the system depicted in FIG. 1;

FIG. 4 illustrates an example system including a forceps housing section of a reconfigurable multi-purpose surgical tool, which can form at least a portion of the system depicted in FIG. 1;

FIG. 5 illustrates an example schematic diagram of system including an electrical configuration of a reconfigurable multi-purpose surgical tool, which can form at least a portion of the system depicted in FIG. 1;

FIG. 6 illustrates an example system including different housing shapes and

configurations of a reconfigurable multi-purpose surgical tool, which can form at least a portion of the system depicted in FIG. 1; and

FIG. 7 illustrates an example method for manufacturing a reconfigurable multi-purpose surgical tool, which can be performed by the system depicted in FIG. 1.

### DETAILED DESCRIPTION

It will be appreciated that for simplicity and clarity of illustration, where appropriate, reference numerals have been repeated among the different figures to indicate corresponding or analogous elements. In addition, numerous specific details are set forth to provide a thorough understanding of the examples described herein. However, it will be understood by those of ordinary skill in the art that the examples described herein can be practiced without these specific details. In other instances, methods, procedures, and components have not been described in detail so as not to obscure the related relevant feature being described. Also, the description is not to be considered as limiting the scope of the examples described herein. The drawings are not necessarily to scale and the proportions of certain parts may be exaggerated to better illustrate details and features of the present disclosure.

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the presently disclosed technology or the appended claims. Further, it should be understood that any one of the features of the presently disclosed technology may be used separately or in combination with other features. Other systems, methods, features, and advantages of the presently disclosed technology will be, or become, apparent to one with skill in the art upon examination of the figures and the detailed description. It is intended that all such additional systems, methods, features, and advantages be included within this description, be within the scope of the presently disclosed technology, and be protected by the accompanying claims.

Further, as the presently disclosed technology is susceptible to examples of many different forms, it is intended that the present disclosure be considered as an example of the principles of the presently disclosed technology and not intended to limit the presently disclosed technology to the specific examples shown and described. Any one of the features of the presently disclosed technology may be used separately or in combination with any other feature. References to the terms “example,” “examples,” and/or the like in the description mean that the feature and/or features being referred to are included in, at least, one aspect of the description. Separate references to the terms “example,” “examples,” and/or the like in the description do not necessarily refer to the same example and are also not mutually exclusive unless so stated and/or except as will be readily apparent to those skilled in the art from the description. For example, a feature, structure, process, step, action, or the like described in one example may also be included in other examples but is not necessarily included. Thus, the presently disclosed technology may include a variety of combinations and/or integrations of the examples described herein. Additionally, all aspects of the present disclosure, as described herein, are not essential for its practice. Likewise, other systems, methods, features, and advantages of the presently disclosed technology will be, or become, apparent to one with skill in the art upon examination of the figures and the description. It is intended that all such additional systems, methods, features, and advantages be included within this description, be within the scope of the presently disclosed technology, and be encompassed by the claims.

Any term of degree such as, but not limited to, “substantially,” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees.

The term “coupled” is defined as connected, whether directly or indirectly through intervening components, and is not necessarily limited to physical connections. The connection can be such that the objects are permanently connected or releasably connected. The terms “comprising,” “including” and “having” are used interchangeably in this disclosure. The terms “comprising,” “including” and “having” mean to include, but not necessarily be limited to the things so described. The term “real-time” or “real time” means substantially instantaneously.

Lastly, the terms “or” and “and/or,” as used herein, are to be interpreted as inclusive or meaning any one or any combination. Therefore, “A, B, or C” or “A, B, and/or C” mean any of the following: “A,” “B,” or “C”; “A and B”; “A and C”; “B and C”; “A, B and C.” An exception to this definition will occur only when a combination of elements, functions, steps or acts are in some way inherently mutually exclusive.

The systems, methods, and devices disclosed herein improve upon previous techniques by providing a reconfigurable multi-purpose surgical tool to perform one or more surgical operations. The reconfigurable multi-purpose surgical tool can include one or more tool housing sections (e.g., modules) formed into a single, integrated tool to house multiple surgical tools, which can be converted between different configurations (e.g., an in-use configuration and a stored position).

In some instances, tools are separated to different sides of the multi-purpose surgical tool based on the function of the tools. For instance, a cautery tool and a scalpel tool are both point-of-function tools, where a single point of contact is made. As such, these types of tools can have tool tips that are narrow and can be arranged in the housing with similar orientations, substantially parallel to each other, and/or extending out a first end of the multi-purpose surgical tool while in the housing. The arrangement of these tools can correspond to how the surgeon's hand moves while using the multi-purpose surgical tool; being able to switch seamlessly from the cautery module to the scalpel module can be beneficial relative to other arrangements, where the device may be rotated to alternate between these tools. In contrast, a forceps tool works with two points of contact on the patient, meaning the forceps tool is wider than the scalpel tool and cautery tool. Accordingly, the forceps tool can be disposed in a forceps module extending from a second end of the multi-purpose surgical tool opposite from the first end. This can help limit line of sight interruption. In other words, limiting the largest of the tools to one side can provide a clearer line of sight.

In some scenarios, the multi-purpose surgical tool includes various features to increase safety. For instance, when the scalpel housing section or module, or the cautery housing section or module are not being used (e.g., by the surgeon), these tools can be retracted into the housing, preventing accidental cutting or burning during surgery. Additionally, accidental activation of the cautery tool's cut or coagulate function can be avoided by preventing the activation buttons from being pressed unless the cautery tool is fully extended. As such, the cautery tool is unable to injure a person in a partially extended state.

Another benefit of this design, in some examples, is the lack of serious tool modification, leading to a lack of standards utilization. Industry tools can be used in the various housing sections disclosed herein with no or minimal modifications (e.g., such as shortening). Accordingly, few if any relevant medical industry standards may apply to the multi-purpose surgical tool.

Additional advantages of the systems discussed herein will become apparent from the detailed description below.

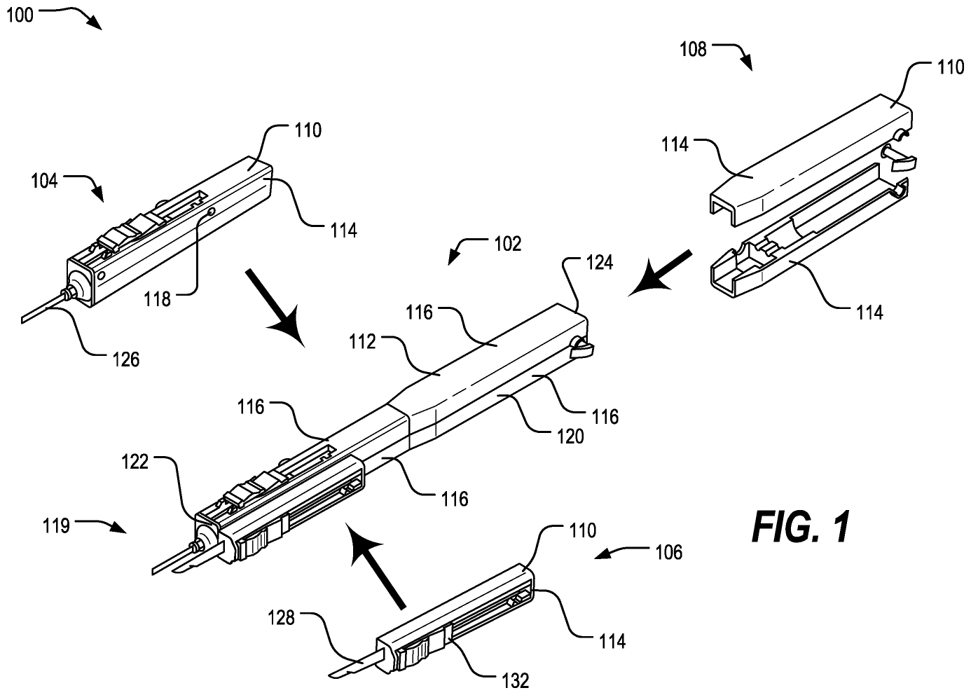

FIG. 1 illustrates an example system 100 including a reconfigurable multi-purpose surgical tool 102 for performing a plurality of surgical operations. The reconfigurable multi-purpose surgical tool 102 can include multiple compartmentalized components aggregated together into a single integrated tool for use in an operating environment (e.g., a clinical environment). For instance, the reconfigurable multi-purpose surgical tool 102 can include a cautery housing section 104, a scalpel housing section 106, a forceps housing section 108, and/or any other type of tool section(s) 110 which can be adjustable into various operational positions and/or stored positions.

In some examples, the reconfigurable multi-purpose surgical tool 102 includes a housing 112. The housing 112 can be formed of the housing portions 114 having various interior compartments, channels and/or mounting surfaces of the different tool section(s) 110. The housing portions 114 forming the different tool section(s) 110 can be manufactured individually or separately before being combined together into the reconfigurable multi-purpose surgical tool 102. Additionally or alternatively, the one or more housing portions 114 of the different tool section(s) 110 can be manufactured as a single housing with continuous exterior surfaces and/or interior chambers. The main housing 112, in some instances, is split into four housing quarters 116 such that components can be placed inside and sealed within the tool section(s) 110. The components and/or housing portions 114 (e.g., the four housing quarters 116) can be joined via plastic welding, which can make the housing 112 very rigid and/or can be less labor intensive than using an adhesive. Once the four housing quarters 116 are connected together, the scalpel housing section 106 can be coupled to the cautery housing section 104 using one or more alignment holes 118 and/or can be welded to the cautery housing section 104.

In some instances, the cautery housing section 104, the scalpel housing section 106, the forceps housing section 108, and any combinations thereof comprise housing portions 114 that can be converted between a plurality of configurations such as a an in-use configuration 119 and/or a stored configuration. At least portions of the surgical tools (e.g., sharp edges, tips, heating elements, etc.) of the different housing portions 114 can be at least partially concealed, contained, stored, or obstructed in the stored configuration. Additionally or alternatively, at least portions of the surgical tools can be at least partially exposed and/or extended from the housing 112 when the surgical tool(s) are in the in-use configuration.

In some examples, the section(s) 110 of the reconfigurable multi-purpose surgical tool 102 can be an elongated housing 120, narrow in the hand like a pencil, with tools at both ends. For instance, a first end 122 of the reconfigurable multi-purpose surgical tool 102 can include the cautery housing section 104 and the scalpel housing section 106. A second end 124 can include the forceps housing section 108. As such, the cautery housing section 104 and the scalpel housing section 106 can extend from a same side, and the forceps housing section 108 can extend from an opposite side, such as 180° in an opposite direction away from the direction in which the cautery housing section 104 and/or the scalpel housing section 106 extend. Furthermore, with the cautery housing section 104 and the scalpel housing section 106 on the same side, these modules can have mechanisms to actuate back and forth in a way that avoids interfering with or hindering the surgeon's line of sight. Moreover, the reconfigurable multi-purpose surgical tool 102 can have a compact configuration in some instances by modifying the cautery tool 126, the scalpel tool 128, and/or the forceps tool 130. For example, the cautery housing section 104 can include an outer plastic shell, which can be modified/removed to expose a circuit. The scalpel tool 128 can be machined to be shorter and/or can have a hole in it to attach to the scalpel housing section 106. The forceps tool 130 can remain unchanged in its integration into the forceps housing section 108. This can be advantageous because the forceps tool 130 can be expensive and is often sterilized and reused due to its high cost. As such, the reconfigurable multi-purpose surgical tool 102 can be configured such that forceps is removably inserted into the forceps housing section 108, used to perform one or more operations, and is removed for cleaning and rues (e.g., during disposal of remaining portions of the reconfigurable multi-purpose surgical tool 102), significantly reducing the cost per tool.

Furthermore, in some instances, the surgical tools in the section(s) 110 of the reconfigurable multi-purpose surgical tool 102 can be controlled by individual, sliding mechanisms 132 that convert the portions of the reconfigurable multi-purpose surgical tool 102 between the stored configuration(s) and the in use configuration(s). For instance, the sliding mechanism 132 can enable the surgeon to extend and withdraw the scalpel handle 128, the bipolar forceps tool 130, or the cautery tool 126 in a controlled and retractable manner. The sliding mechanism 132 can ride on top of a set of teeth and can have a slider button and/or a locking button. To remove any unwanted up and down movement the slider can be glued or otherwise affixed to the scalpel tool 128. The lock can stop movement and lock the scalpel tool 128 into place and can be pulled out from the slider piece to release the scalpel tool 128. The locking slider can then be pushed in back when movement is desired. This sliding mechanism 132 can provide a smooth slide in both direction without getting caught or dragging. As discussed in greater detail below, both the cautery tool 126 and the scalpel tool 128 can use the same sliding mechanism 132 and/or similar sliding mechanisms with slight variations. For instance, the cautery tool 126 can have a sliding mechanism built into the holster, whereas the scalpel tool 128 can have a separate piece that attaches to the sliding mechanism 132. Moreover, the cautery tool 126 and the scalpel tool 128 can both use a similar or identical locking mechanism.

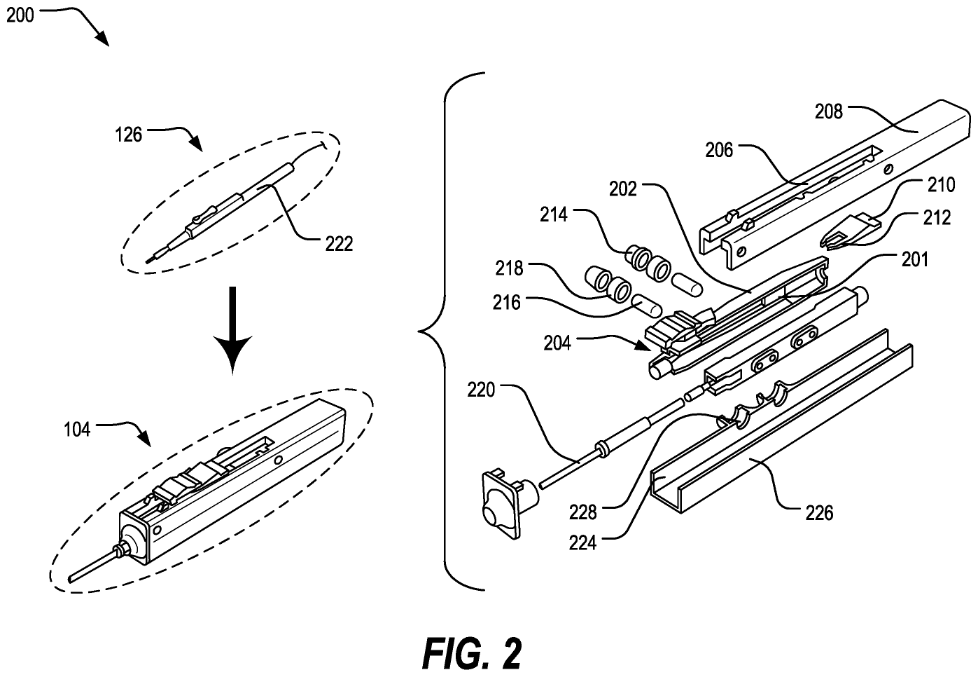

FIG. 2 illustrates an example system 200 including the cautery housing section 104 of the reconfigurable multi-purpose surgical tool 102, which can form at least a portion of the system depicted in FIG. 1.

In some examples, during assembly, a cautery circuit 201 is glued into a cautery outer shell 202 which is the adapter between the cautery circuit and the control system 204. The cautery outer shell 202 can slide back and forth on a toothed track 206 which can be seen at the top of an upper housing portion 208. In some instances, the track 206 can have only two teeth positions, one for the deployed position and one for the fully retracted position, which can improve safety since these are the safest positions for the cautery tool 126. Once the desired position is reached, the cautery outer shell 202 (e.g., the shell assembly) can be locked in place with a cautery lock 210. The cautery lock 210 can have a small cube, protrusion, or other bulging portion molded to a flexible arm 212 which, when pulled, spreads the mechanism and locks it into one of the teeth. Once deployed the cautery tool 126 can perform cut and coagulate functions, which can be activated using buttons of a Monopolar Cautery Pencil (e.g., original buttons). These buttons 214 can work the same as the original cautery, but have a longer, extended shaft 216 glued on the end to make up the larger gap between the buttons on the reconfigurable multi-purpose surgical tool 102 and the buttons on the sliding cautery circuit 201, for instance, to retrofit the cautery pencil by integrating it into the reconfigurable multi-purpose surgical tool 102. Furthermore, a small piece of foam 218 can be used as a retainer to push the button and shaft assembly to its furthest position and allow the cautery circuit 201 to slide smoothly.

In some instances, the cautery pencil 220 can have a plastic outer shell 222 removed to lessen the size of its housing. The cautery pencil 220 can include only a small internal circuit inside controlling its functions, so retrofitting the cautery pencil 220 to only extract the internal circuit and wiring for integration into the reconfigurable multi-purpose surgical tool 102 can reduce the size of the reconfigurable multi-purpose surgical tool 102 by more efficiently using space. The extracted cautery circuit 201 can be attached to the slidable cautery outer shell 202. Additionally, a side 224 of the main outer housing 226 can contain holes 228 to place the buttons 214 which interact with the cautery circuit 201, turning the cut and coagulate functions provided by the cautery housing section 104 on and off. The buttons 230 can have a shape configured to fit within the frame.

In some examples, a benefit of the technology disclosed herein is the lack of serious tool modification, which can lead to a lack of standards utilization. For instance, some instances have minimal modification limited to replacing the cautery tool housing and shortening the tools. As such, medical device standards are not relevant to the reconfigurable multi-purpose surgical tool 102.

In some examples, the reconfigurable multi-purpose surgical tool 102 includes unique features to increase safety. The cautery housing section 104 and the scalpel housing section 106 can be transitioned to the retracted positions when not being used (e.g., by the surgeon). In the retracted position, the scalpel blade and cautery tip can be pulled into and at least partially enclosed by the housing 112. This can prevent accidental cutting or burning during surgery. An additional safety feature can prevent accidental activation of the cautery's cut or coagulate function, for instance, by preventing the activation buttons from being pressed unless the cautery is in a fully extended (e.g., via one or more switches and/or sensors disposed in the cautery circuit 201). This means that unless the surgeon has fully extended the cautery in the device, the cautery cannot accidentally injure a person. This is a simple yet functional solution.

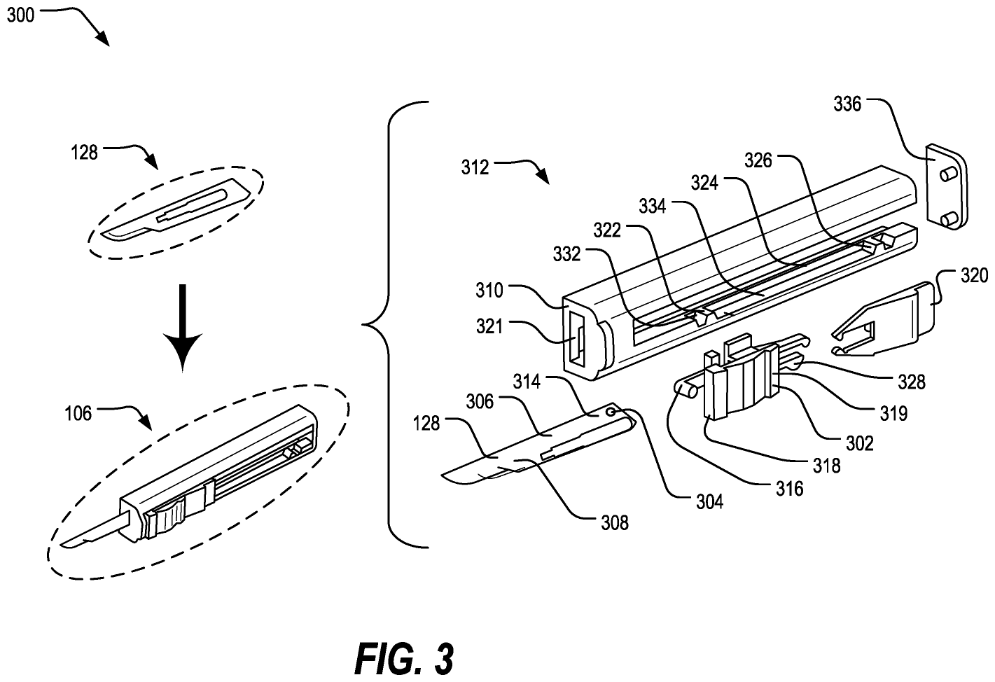

FIG. 3 illustrates an example system 300 including the scalpel housing section 106 of the reconfigurable multi-purpose surgical tool 102, which can form at least a portion of the system depicted in FIG. 1.

In some instances, the scalpel housing section 106 functions can be similar to those of the cautery housing section 104. The scalpel housing section 106 can include a slider 302 which attaches to a hole 304 machined into a modified scalpel handle 306. In addition to the pin and hole attachment the scalpel tool 128 can also be glued to the slider 302. The slider 302 with the scalpel 308 attached can be disposed in the scalpel housing 310, and the endcap can be welded on to the end of the scalpel housing 310. This entire scalpel assembly 312 can then be joined using the 2 pins on the scalpel housing 310 to weld or otherwise affix the scalpel housing 310 section 106 onto the main housing (e.g., at the side of the cautery housing section 104).

In some examples, the scalpel housing section 106 attaches to the side of the reconfigurable multi-purpose surgical tool 102 at a same end or a same half as the cautery housing section 104. The scalpel tool 128 can have a modified, reduced handle 314 length to integrate the scalpel tool 128 into the scalpel housing section 106 via a retrofit process. The handle 314 of the scalpel tool 128 can have the attachment hole 304 for attaching the scalpel tool 128 to the slider 302 using a peg 316 protruding from the slider 302. The scalpel tool 128 can connect to the slider 302 at a first side 318 opposite a second side 319 to which the slide lock 320 attaches. The scalpel housing section 106 can be formed into the reconfigurable multi-purpose surgical tool 102 on either side of the cautery housing section 104. In some instances, the reconfigurable multi-purpose surgical tool 102 is ambidextrous in that it can be configured for use with a left hand or a right hand. The scalpel housing section 106 can be reconfigurable such that it can be placed onto and/or removed from either side of the cautery housing section 104 or further repositioned at various locations on the scalpel housing section 106 to adapt to the hand shape/size of the user and/or provide the ambidextrous usability.

In some scenarios, the reconfigurable multi-purpose surgical tool 102 provides for using a standardization of blade attachments without requiring more custom-made parts than necessary, which improves the ease of manufacturing and lowers costs. The scalpel 308 can slide in and out of an opening 321 at an end of the scalpel housing section 106 to transition between a stored position and an operating position. The scalpel housing section 106 can include a first tooth 322 at a first end of a sliding channel 324 and a second tooth 326 at a second end of the sliding channel 324 opposite the first end. The first tooth 322 and the second tooth 326 can correspond to a locking member 328 (e.g., a flexible post or bulge) extending from the slider 302, such that bending the locking member 328 toward the channel causes the locking member 328 to slide in and out of a locking gap 332 (e.g., separated by a sliding gap 334) defining the locking positions. An end cap 336 can enable a surgeon to insert the scalpel 308 and then be able to seal off the rear of the scalpel housing 310, preventing the scalpel 308 from sliding out. The scalpel 308 can be a #10 or #15 and the size can be interchanged as desired.

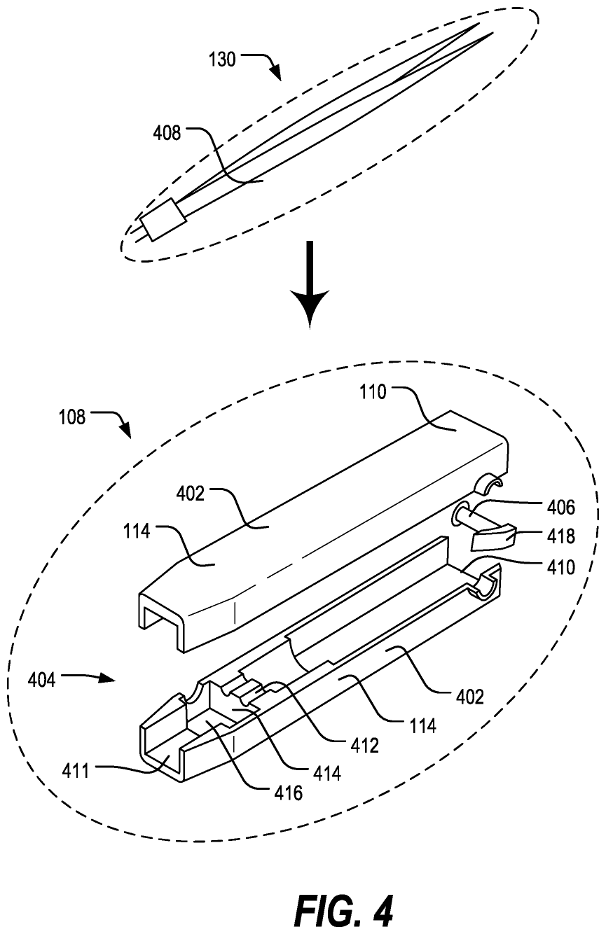

FIG. 4 illustrates an example system 400 including a forceps housing section 108 of a reconfigurable multi-purpose surgical tool 102, which can form at least a portion of the system 100 depicted in FIG. 1.

In some examples, the forceps housing section 108 is simpler and can include only three pieces such as two housing halves 402 of the forceps housing 404, and an actuator pin 406. The forceps 408 can be stationary with portion (e.g., a half) of the forceps 408 extending out an opening 410 at a second end of the reconfigurable multi-purpose surgical tool 102. This can save space inside the reconfigurable multi-purpose surgical tool 102 and/or reduce an overall length of the housing 112. The actuator pin 406 can be pressed to abut and push against one of the forceps arms to actuate the forceps 408, as fingers would on normal forceps. The forceps housing 404 can also include a connector recess 411 forming extra space to house an electrical connector attached to the electrical wire(s) of the reconfigurable multi-purpose surgical tool 102. Pins along with the internal cylinder can hold the forceps tightly in place, while also being completely removable at the end of usage. Additionally, one or more grooves 412 (e.g., two grooves) can be formed into one or more resting mounts 414 extending from an inner surface 416 of the forceps housing 404 for positioning the forceps tool 130 within the forceps housing 404.

In some instances, the forceps 408 are inserted into the device to a specified length to reduce the overall length of the reconfigurable multi-purpose surgical tool 102 while still retaining multiple advantages. The forceps 408 can be inserted into the standard insertion that will be enclosed within the device by adhesives attached to a mold printed onto the frame. For using the forceps housing section 108, an operator of the reconfigurable multi-purpose surgical tool 102 can grasp the first end 122 (e.g., the cautery housing section 104 and/or the scalpel housing section 106) with one hand while grasping the second end 124 and operating the actuator pin 406 with the other hand to avoid issues of weight distribution. The actuator pin 406 can be pressed by the surgeon's finger to push an end 418 of the actuator pin 406 through the housing and into the forceps 408, closing the forceps 408 as needed. Additionally or alternatively, the housing materiel of the forceps housing 404 itself can be at least partially flexible (e.g., formed of rubber or silicon), such that the housing 404 can be squeezed to compress the forceps 408.

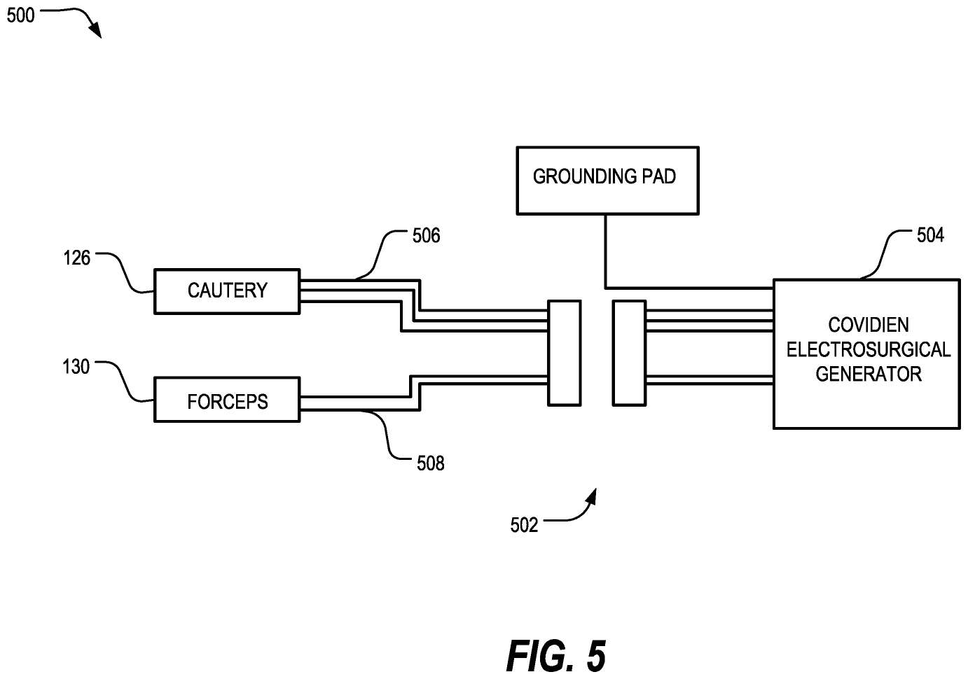

FIG. 5 illustrates an example schematic diagram of system 500 including an electrical configuration 502 of the reconfigurable multi-purpose surgical tool 102, which can form at least a portion of the system depicted in FIG. 1.

In some examples, an electrical surgical unit 504 will supply to the cautery tool 126 and the forceps tool 130 by the power cables being connected from the unit which can run through a circular hole cut into the side of the product and controlled by a grommet and wire loom to reduce wire management needs and prevent debris getting within the product. The reconfigurable multi-purpose surgical tool 102 can have one or more first cables 506 providing power to the cautery tool 126 and one or more second cables 508 providing power to the forceps tool 130. A single waterproof connector can be spliced to connect the cautery tool 126 and the forceps tool 130 to the plugs used in an electrosurgical generator (e.g., the Covidien Electrosurgical Generator). As such, the reconfigurable multi-purpose surgical tool 102 can have a short harness that can be packaged with the reconfigurable multi-purpose surgical tool 102 and can be disposable, as opposed to the entire length of harness being needed to plug the reconfigurable multi-purpose surgical tool 102 into the into the electrosurgical generator.

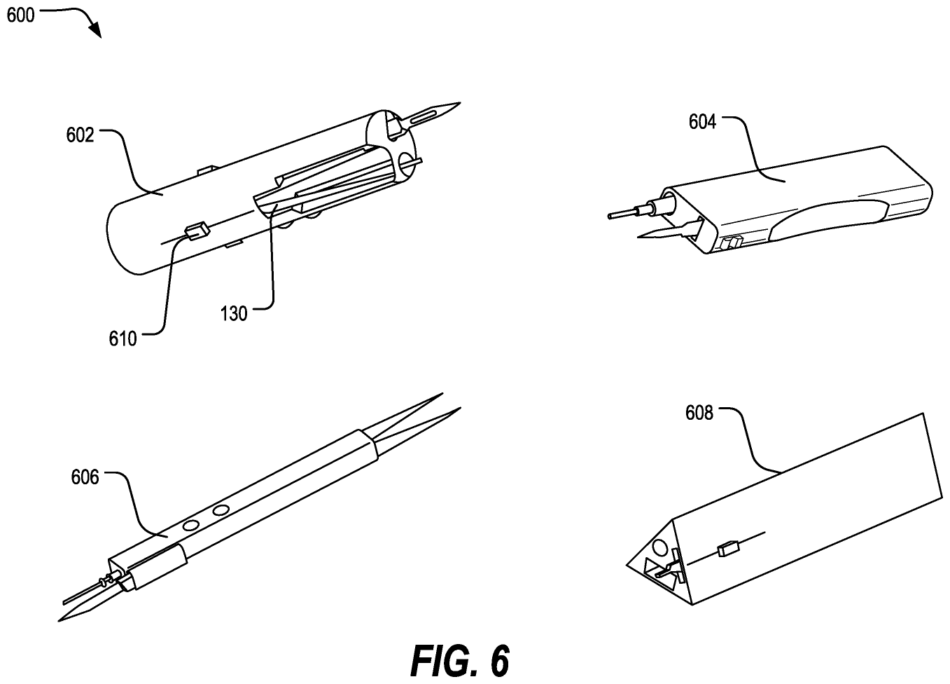

FIG. 6 illustrates example system(s) 600 including different housing shapes and module configurations, which can form at least a portion of the system 100 depicted in FIG. 1.

For instance, in some examples, the reconfigurable multi-purpose surgical tool 102 can have a circular housing 602, a rectangular housing 604, a square housing 606, and/or a triangular housing 608. Furthermore, in some instances, the forceps tool 130 can extend from a same side as the cautery tool 126 and/or the scalpel tool 128. For instance, the forceps tool 130 can have a forceps slider 610 for pushing the forceps tool 130 out one or more openings on a surface from which the scalpel tool 128 and/or the cautery tool 126 also extend.

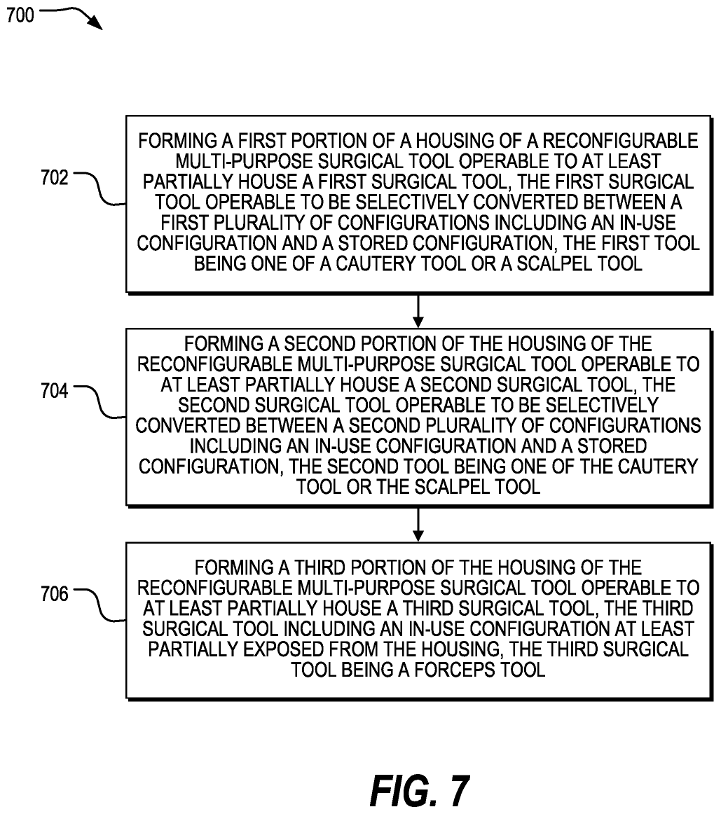

FIG. 7 illustrates an example method 700 for forming or manufacturing the reconfigurable multi-purpose surgical tool 102 depicted in FIGS. 1-6.

At operation 702, the method 700 can include forming a first portion of a housing of a reconfigurable multi-purpose surgical tool operable to at least partially house a first surgical tool. The first surgical tool may be operable to be selectively converted between a first plurality of configurations including an in-use configuration and a stored configuration, the first tool being one of a cautery tool or a scalpel tool. At operation 704, the method 700 can include forming a second portion of the housing of the reconfigurable multi-purpose surgical tool operable to at least partially house a second surgical tool, the second surgical tool operable to be selectively converted between a second plurality of configurations including an in-use configuration and a stored configuration, the second tool being one of the cautery tool or the scalpel tool. At operation 706, the method can include forming a third portion of the housing of the reconfigurable multi-purpose surgical tool operable to at least partially house a third surgical tool, the third surgical tool including an in-use configuration at least partially exposed from the housing, the third surgical tool being a forceps tool.

It is to be understood that the specific order or hierarchy of steps in the method depicted in FIG. 7 and throughout this disclosure are instances of example approaches and can be rearranged while remaining within the disclosed subject matter. For instance, any of the operations depicted in FIG. 7 and throughout this disclosure can be omitted, repeated, performed in parallel, performed in part, performed in a different order, and/or combined with any other of the operations depicted in FIG. 7 and/or throughout this disclosure, or portions thereof.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined differently in various implementations of the disclosure or described with different terminology. These and other variations, modifications, additions, and improvements may fall within the scope of the disclosure as defined in the claims that follow.

What is claimed is:

1. A reconfigurable multi-purpose surgical tool comprising:
a housing;
a first portion of the housing operable to at least partially house a first surgical tool, the first surgical tool operable to be selectively converted between a plurality of configurations, the plurality of configurations including a first in-use configuration and a stored configuration, the stored configuration including a first surgical tool portion of the first surgical tool being at least partially contained by the housing, the first in-use configuration being when the first surgical tool portion is at least partially exposed from the housing; and
a second portion of the housing operable to at least partially house a second surgical tool, the second surgical tool operable to include at least a second in-use configuration, the second in-use configuration being when the second surgical tool is at least partially exposed from the housing.

2. The reconfigurable multi-purpose surgical tool of claim 1, wherein:
the first portion of the housing is a cautery housing section;
the first surgical tool is a cautery tool;
the second portion of the housing is a forceps housing section; and
the second surgical tool is a forceps tool.

3. The reconfigurable multi-purpose surgical tool of claim 1, further comprising:
a third portion of the housing operable to at least partially house a scalpel tool.

4. The reconfigurable multi-purpose surgical tool of claim 1, wherein the first surgical tool extends from an opposite end of the housing relative to the second surgical tool when the first surgical tool is in the first in-use configuration.

5. The reconfigurable multi-purpose surgical tool of claim 1, wherein the first surgical tool is operable to be in the first in-use configuration when the second surgical tool is in the second in-use configuration.

6. The reconfigurable multi-purpose surgical tool of claim 1, wherein the second portion of the housing includes an internal mounting surface operable to securely position the second surgical tool in the second portion of the housing.

7. The reconfigurable multi-purpose surgical tool of claim 1, further comprising:
a tool slider with a locking mechanism operable to cause the first surgical tool to be converted between the plurality of configurations.

8. The reconfigurable multi-purpose surgical tool of claim 1, further comprising:
an electrical connector operable to connect at least one of the first surgical tool or the second surgical tool to an electrosurgical generator.

9. The reconfigurable multi-purpose surgical tool of claim 1, wherein:
the first portion of the housing is a cautery housing section;
the first surgical tool is a cautery tool;
the second portion of the housing is a scalpel housing section; and
the first surgical tool is a scalpel tool.

10. The reconfigurable multi-purpose surgical tool of claim 9, wherein the second surgical tool is operable to be selectively converted between a second plurality of configurations, the second plurality of configurations including the second in-use configuration and a second stored configuration, the second stored configuration including a second surgical tool portion of the second surgical tool being at least partially contained within the housing.

11. A reconfigurable multi-purpose surgical tool comprising:
a housing;
a first portion of the housing operable to at least partially house a first surgical tool, the first surgical tool operable to be selectively converted between a plurality of configurations, the plurality of configurations including an in-use configuration and a stored configuration, the stored configuration including a first surgical tool portion of the first surgical tool being at least partially contained by the housing, the in-use configuration being when the first surgical tool portion is extended at least partially from a first end of the housing; and
a second portion of the housing operable to at least partially house a second surgical tool, the second surgical tool, having a configuration extending from a second end of the housing opposite the first end.

12. The reconfigurable multi-purpose surgical tool of claim 11, wherein:
the first portion of the housing is a cautery housing or a scalpel housing;
the first surgical tool is a cautery tool or a scalpel tool;
the second portion of the housing is a forceps housing; and
the first surgical tool is a forceps tool.

13. The reconfigurable multi-purpose surgical tool of claim 11, further comprising:
a slider assembly with a first surgical tool locking member operable to engage with a slide channel along the housing, the first surgical tool being a cautery tool.

14. The reconfigurable multi-purpose surgical tool of claim 13, further comprising:
a third surgical tool being a scalpel tool; and
a second slider assembly coupled to the scalpel tool, the second slider assembly operable to move the scalpel tool between an extended position and a retracted position, the second slider assembly including a second tool locking member operable to engage with a second slide channel along the housing.

15. The reconfigurable multi-purpose surgical tool of claim 14, wherein the third surgical tool is extendible from the first end of the housing in a direction parallel to the first surgical tool when the third surgical tool is in a third in use configuration.

16. A reconfigurable multi-purpose surgical tool comprising:
a housing;
a first portion of the housing operable to at least partially house a first surgical tool, the first surgical tool operable to be selectively converted between a first plurality of configurations, the first plurality of configurations including a first in-use configuration and a stored configuration, the stored configuration including a first surgical tool portion of the first surgical tool being at least partially contained by the housing, the first in-use configuration being when the first surgical tool is at least partially exposed; and
a second portion of the housing operable to at least partially house a second surgical tool, the second surgical tool operable to be selectively converted between a second plurality of configurations, the second plurality of configurations including a second in-use configuration and another stored configuration, the second stored configuration including a second surgical tool portion of the second surgical tool being at least partially contained by the housing, the second in-use configuration being when the second surgical tool portion is at least partially exposed at a same end of the housing as the first surgical tool.

17. The reconfigurable multi-purpose surgical tool of claim 16, wherein:
the first portion of the housing is a cautery housing section;
the first surgical tool is a cautery tool;
the second portion of the housing is a scalpel housing section; and
the first surgical tool is a scalpel tool.

18. The reconfigurable multi-purpose surgical tool of claim 17, further comprising:
one or more power buttons disposed along a sidewall of the cautery housing section of the housing, the one or more power buttons operable to control a power supply to a control circuit of the cautery tool.

19. The reconfigurable multi-purpose surgical tool of claim 17, further comprising:
a third portion of the housing operable to at least partially house a third surgical tool, the third surgical tool being a forceps tool.

20. The reconfigurable multi-purpose surgical tool of claim 19, further comprising:
an actuator button disposed at a side wall of the third portion of the housing, the actuator button operable to apply a compressive force to the forceps tool when the forceps tool is in a third in-use configuration.
